(19) United States
(12) Patent Application Publication
(10) Pub. No.: US 2024/0156779 A1
(43) Pub. Date: May 16, 2024

(54) SMALL MOLECULE ALLOSTERIC MODULATORS OF CLASS B GPCR, THE PTHR, AND METHOD TO IDENTIFY THEM

(71) Applicant: University of Pittsburgh - Of the Commonwealth System of Higher Education, Pittsburgh, PA (US)

(72) Inventors: Ieva Sutkeviciute, Pittsburgh, PA (US); Jean-Pierre Vilardaga, Pittsburgh, PA (US); Jiyoung Lee, Wexford, PA (US); Ivet Bahar, Wexford, PA (US); Burak Kaynak, Pittsburgh, PA (US)

(73) Assignee: University of Pittsburgh - Of the Commonwealth System of Higher Education, Pittsburgh, PA (US)

(21) Appl. No.: 18/277,765

(22) PCT Filed: Mar. 9, 2022
(86) PCT No.: PCT/US2022/019464

§ 371 Date: Aug. 17, 2023

Related U.S. Application Data
(60) Provisional application No. 63/159,338, filed on Mar. 10, 2021.

(51) Int. Cl. A61K 31/401

(52) U.S. Cl. CPC A61K 31/401

(57) ABSTRACT

Disclosed herein are methods for treating hyperparathyroidism, osteoporosis, or cancer cachexia, or inhibiting abnormally increased white adipose tissue browning, or decreasing the risk of a kidney stone in a subject, comprising administering to a subject in need thereof a therapeutically effective amount of a negative allosteric modulator of parathyroid hormone (PTH) type 1 receptor (PTHR) signaling.

### CROSS REFERENCE TO RELATED APPLICATION

This application claims the benefit of the earlier filing date of U.S. provisional patent application No. 63/159,338, filed Mar. 10, 2021, which is incorporated herein by reference in its entirety.

### ACKNOWLEDGMENT OF GOVERNMENT SUPPORT

This invention was made with government support under grant numbers TR⁰⁰¹⁸⁵⁷ and DK116780 awarded by the National Institutes of Health. The government has certain rights in the invention.

### BACKGROUND

G protein-coupled receptors (GPCRs) are the most abundant cell membrane receptors in the human genome for extracellular (EC) stimuli such as hormones and neurotransmitters, and the targets of ˜35% of currently approved clinical drugs. While they are essential drug discovery targets, only 18% of the total estimated pool of >1000 human GPCRs are targeted, with ˜94% of drugs acting through class A GPCRs (rhodopsin-like receptors). The initial identification of a lead compound as a drug candidate is a critical step in the drug discovery process. Current strategy for GPCR drug discovery relies on screening large libraries, either via High-Throughput Screening (HTS) of selected drug-like molecule libraries, or a recent structure-guided in silico screening of virtual compound libraries and/or docking simulations to identify the best (highest affinity) fit to the target protein structure. The latter has experienced a substantial advancement with the recent development of an ultra-large make-on demand drug-like compound library and the first successful example of using this new tool. Nonetheless, both approaches hold two substantial caveats: 1) due to the astronomical numbers (˜10²³-10⁶⁰) of possible drug-like molecules, only a limited number of compounds are selected to build libraries for testing in HTS assays, therefore results depend on subjective library selection that may not lead to successful lead compound discovery, and 2) virtual screening requires not only the high-resolution structure of the target receptor but also knowledge of one or more druggable sites in line with the conformational flexibility of the target, including allosteric sites to be selectively targeted for modulating selective interactions. These caveats may at least partially be overcome for class A GPCRs because: 1) many receptors of this class bind the endogenous ligands that are small molecules, such as amino acid derivatives or lipids, allowing for the design of compound libraries for HTS by selecting drug-like molecules with structures similar to those of the endogenous ligands, and 2) the orthosteric ligand binding sites of class A GPCRs are mostly compact pockets, making them well-suited docking sites in virtual screening approach. This partially explains the aforementioned success of drug discovery for class A GPCRs.

Of large interest is the development of orally available small molecule drugs for class B GPCRs (secretin-like receptors that bind moderately sized peptide hormones) to treat numerous metabolic disorders including diabetes, bone and mineral ion disorders, cardiovascular disease, neurodegeneration, and psychiatric disorders. The distinguishing structural feature of class B GPCRs is a large EC domain (ECD), which captures the endogenous peptide hormone ligands by their C-terminal part, leading to the insertion of the N-terminal portion of the peptide hormone deep into transmembrane (TMD) domain's (TMD) EC cavity and subsequent receptor activation. Such hormone binding mode results in a highly extended endogenous ligand recognition and binding region spanning the entire ECD and reaching all the way down to the mid-region of the TMD. Undeniably, the highly extended endogenous ligand binding region coupled to the difficulty in perturbing large protein-protein interfaces by small organic molecules, constitute a major hurdle in the process of discovering non-peptidic small molecule drug candidates for class B GPCRs. Currently, ubrogepan —an orally administered calcitonin gene-related peptide receptor antagonist for the treatment of acute migraine—is the only U.S. Food and Drug Administration (FDA)-approved small molecule drug for a class B GPCR. Two other small molecule drug candidates have successfully completed phase 2 clinical trials: TTP273, orally available glucagon-like peptide 1 receptor (GLP1R) agonist for the treatment of type 2 diabetes mellitus; and Verucerfont, orally available corticotrophin-releasing factor-1 (CRF-1) receptor small molecule antagonist for the treatment of post-traumatic stress disorder or alcoholism. The vast majority of the rest of the approved drugs targeting class B GPCRs are either native or modified endogenous peptides. However, the peptidic nature of a drug has several disadvantages including costs and low patient's adherence to the therapy as they require frequent injections due to oral inaccessibility and short plasma half-lives. Furthermore, overshoot or undershoot of peptide injections can cause unwanted side-effects.

Therefore, the identification of small molecules may lead to discovery of allosteric modulators, which may present particular advantages of low cost, oral accessibility, and potential to avoid side-effects as they can alter and/or fine-tune receptor signaling induced by endogenous ligands.

### SUMMARY

Disclosed herein are methods for treating hyperparathyroidism, osteoporosis, or cancer cachexia in a subject, comprising administering to a subject in need thereof a therapeutically effective amount of a compound, or a pharmaceutically acceptable salt thereof, of formula I:

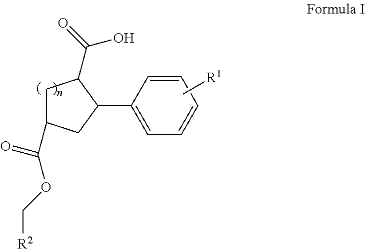

wherein R¹ is hydrogen, alkyl, substituted alkyl, alkoxy, substituted alkoxy, hydroxy, or halogen; R² is phenyl, substituted phenyl,

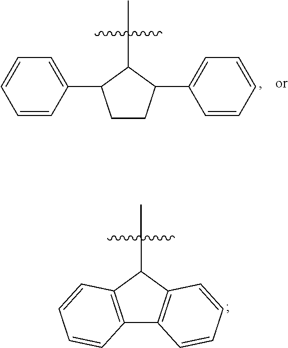 [SMILES: *C1C(c2ccccc2)CCC1c1ccccc1 ; *C1c2ccccc2-c2ccccc21]

and
n is 1 to 3.

Also disclosed herein are methods for treating hyperparathyroidism, osteoporosis, or cancer cachexia in a subject, comprising administering to a subject in need thereof a therapeutically effective amount of a compound, or a pharmaceutically acceptable salt thereof, of formula II:

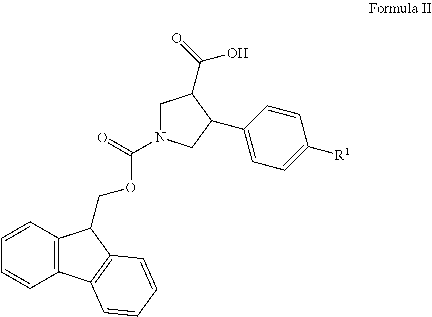

wherein R¹ is hydrogen, alkyl, substituted alkyl, alkoxy or substituted alkoxy.

Also disclosed herein is a method for inhibiting increased white adipose tissue browning in a subject, comprising administering to a subject in need thereof a therapeutically effective amount of a compound, or a pharmaceutically acceptable salt thereof, of formulae I or II.

Also disclosed herein is a method for decreasing the risk of a kidney stone in a subject, comprising administering to a subject in need thereof a therapeutically effective amount of a compound, or a pharmaceutically acceptable salt thereof, of formulae I or II.

Also disclosed herein is a method for negatively modulating signaling and in vivo function of a parathyroid hormone (PTH) type 1 receptor (PTHR), comprising contacting the PTHR with a compound, or a pharmaceutically acceptable salt thereof, of formulae I or II.

Also disclosed herein is a method for negatively modulating signaling and in vivo function of a parathyroid hormone (PTH) type 1 receptor (PTHR), comprising contacting the PTHR with a compound, or a pharmaceutically acceptable salt thereof, of formulae I or II.

Also disclosed herein is a method for negatively allosteric modulating signaling of a parathyroid hormone (PTH) type 1 receptor (PTHR), comprising contacting the PTHR with a compound, or a pharmaceutically acceptable salt thereof, of formulae I or II.

Also disclosed is a method for treating hyperparathyroidism, osteoporosis, or cancer cachexia, or inhibiting abnormally increased white adipose tissue browning, or decreasing the risk of a kidney stone in a subject, comprising administering to a subject in need thereof a therapeutically effective amount of a negative allosteric modulator of parathyroid hormone (PTH) type 1 receptor (PTHR) signaling.

The foregoing will become more apparent from the following detailed description, which proceeds with reference to the accompanying figures.

### BRIEF DESCRIPTION OF THE DRAWINGS

FIGS. 1A-1E. ESSA points to an extracellularly-exposed pocket as an essential site that can potentially alter the allosteric dynamics of PTHR upon ligand binding.

FIG. 1A. Distribution of ESSA z-scores for PTHR active conformer in the presence (red curve) or absence (blue curve) of PTH. Peaks indicate the essential sites. Residue ranges of the PTH, EC domain and TM helices are indicated along the upper abscissa, also delimited by different shades in the graph. TM residues E180, R181, D185, Y191, D241 and Y245 on TM1 and TM2 exhibit z-score above the threshold value of 1.0 (without peptide) (see Methods). Among them, R181 and Y245 exhibit peaks in both structures. Other peaks correspond to loop regions. Thus, R181 and Y245 stand out as essential TM sites that can modulate the global dynamics.

FIGS. 1B-1C. Same results as in FIG. 1A, illustrated by color-coded diagrams side (top) and cytoplasmic-facing (bottom) views. FIG. 1B shows a global hinge site (yellow dashed line) that corresponds to the blue peaks in FIG. 1A. High-to-low scores are color-coded from red-to-blue. The peptide PTH is shown in magenta ribbon in FIG. 1C. A significant increase in sensitivity is observed at the G protein binding region in the presence of PTH.

FIG. 1D. Two hydrophobic pockets (wheat and gray) determined by Fpocket, surrounded by five of the essential residues detected by ESSA (without the peptide).

FIG. 1E. Structural elements identified by ESSA to potentially alter the essential dynamics of the receptor, color coded by ESSA score from red (z-score>3) to blue (z-score close to zero). The analysis was performed for PTH-bound PTHR structure in an active state (PDB id: 6nbf) after modeling the missing loops.

FIGS. 2A-2B. Identification of druggable sites and drug-like small molecules targeting PTHR.

FIG. 2A Summary of a 6-step computational protocol toward identification of small molecule modulators of PTHR activity. The pipeline comprises three major components: druggability simulations using DruGUI (left; blue box); detection of a high-affinity essential site and pharmacophore modeling using Pharmmaker (middle; yellow box), and virtual screening (VS) of one or more pharmacophore models (PM) against libraries of small molecules using Pharmit (right, green box). See text for details. The figure illustrates how two hits, designated as Pitt8 and Pitt12 were derived using the PM deduced from DruGUI.

FIG. 2B. Effect of computationally identified small molecules on PTH-induced cAMP production in HEK293 cells stably expressing the recombinant human PTHR. The bars graph represents the area under the curve (AUC) of cAMP time-courses with 1 nM PTH with or without 10 μM of Pitt compound. Data were normalized to PTH-alone generated AUC and expressed in %-values. Red bars represent negative modulation; MolPort IDs are changed to Pitt #s for convenience. Dotted lines are arbitrary thresholds of 1.5-fold above or below PTH-alone i mean values±s.d. of N=2-3 independent experiments.

FIGS. 3A-3F. Actions of selected compounds on PTH signaling.

FIG. 3A, FIG. 3B. Concentration-response curves for cAMP production by PTH alone or together with a range of concentrations (1-1000 μM) of Pitt8, or Pitt12. Data are mean±s.e.m. of N=4-5 independent experiments (FIG. 3A). Chemical structures of Pitt8 and Pitt12 (FIG. 3B).

FIG. 3C. Averaged cAMP time-courses following brief stimulation with 1 nM PTH without (Ctrl, black) or with 10 μM Pitt molecules measured by FRET changes from HEK293 cells stably expressing PTHR and a FRET-based cAMP sensor Epac^{CFP/YFP}. The bar graph represents the quantitation of cAMP responses by measuring the area under the curve (A.U.C.) from 0 to 20 min. Data are mean±s.e.m. of N=3 experiments with n=26-32 cells/experiments and with the averaged value of the control (DMSO) set to 100.

FIG. 3D. Same experiments as in FIG. 3C performed in cells expressing the dominant-negative dynamin mutant (DynK44A) tagged with RFP.

FIG. 3E. Time course of β-arrestin 2 interaction with PTHR measured by FRET in HEK293 cells transiently expressing PTHR^{CFP} and βarr-2^{YFP} following brief stimulation with 10 nM PTH without (Ctrl) or with 10 μM Pitt molecules. Cells were continuously perfused with control buffer or PTH (horizontal bar). Time course experiments were continuously perfused with control buffer or PTH alone or together with a Pitt molecule (horizontal bar). Data are the mean±s.e.m. of N=3 independent experiments with n=28-32 cells/experiment.

FIG. 3F. Time courses of Ca²⁺ release in response to PTH (100 nM) with or without Pitt molecules (10 μM) in live HEK-293 cells expressing recombinant PTHR.

P values were assessed by two-tailed Student's t-test with *P<0.05, **P<0.005.

FIGS. 4A-4C. In vivo action of Pitt12. Serum Ca²⁺ (sCa²⁺) and phosphate (sPi) levels, as well as BUN/creatine ratio were measured 3 hrs after injections of vehicle (Veh), Pitt12 (20 μmole/kg), PTH1-34 (40 μg/kg), or Pitt12+PTH1-34. N=5-10 mice/group; mean±s.e.m. P values were assessed by one-way ANOVA with Dunnett test. *P values are indicated.

FIGS. 5A and 5B. Construction of pharmacophore model (PM) composed of a hydrogen bond acceptor, a negative ion and two hydrophobic sites (spheres), based on the observed preferential positions, and overlay of a hit compound Pitt12 (aquamarine sticks, extracted from the ZINC database) and the PM.

FIG. 6A. Structural docking of Pitt12 in pink, with PTHR from druggability simulation in dark green and PTH in cyan. Top, top view from extracellular side. Bottom, side view.

FIG. 6B. Insertion of the 9H-fluorene moiety into a hydrophobic patch of TM1 and TM7 residues. Polar interaction between Pitt12 and PTH-His14 is also displayed.

FIG. 6C. Other nearby residues/interactions with Pitt12, including with Leu15 on PTH.

FIG. 6D-6F. Insertion of the benzene moiety into a hydrophobic patch consisting of PTHR (TM1-F184; TM2-L244, Y245, A248) and PTH (Leu 11, Leu15) residues

FIG. 6G. Chemical structure of Pitt12, Pitt41 and Pitt42.

FIG. 6H. Their effects (10 μM) on PTH-mediated cAMP in HEK293 cells stably expressing the PTHR. mean±s.d. of N=2 experiments and n=15-20 cells/exp).

FIGS. 7A-7D. The effect of small molecules on PTH-induced cAMP generation in HEK293 cells expressing PTHR. Left panels show averaged cAMP time courses induced by stimulation with PTH (1 nM) in the absence (black) or in presence (red) of indicated small molecule (10 μM). Black horizontal bar marks the duration of PTH stimulus. Right panels show area under the curve (AUC) plots of corresponding cAMP time courses. Data were normalized to the maximal forskolin induced cAMP response, which is set to 100%. Error bars represent the mean values±s.d.

FIG. 8. Top graph, 1 nM PTH-induced cAMP time courses in the absence (black) or presence of 10 μM Pitt12 (pink), Pitt41 (green) or Pitt42 (orange), in HEK293 cells expressing PTHR; the horizontal bar mark duration of PTH stimulus. Corresponding area under the curve (AUC) bar-graphs are shown in the bottom graph. Data were normalized to the maximal forskolin induced cAMP response, set to 100%. Error bars represent the mean values±SE.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

The term “alkoxy” refers to a straight, branched or cyclic hydrocarbon configuration and combinations thereof, including from 1 to 20 carbon atoms, preferably from 1 to 8 carbon atoms (referred to as a “lower alkoxy”), more preferably from 1 to 4 carbon atoms, that include an oxygen atom at the point of attachment. An example of an “alkoxy group” is represented by the formula —OR, where R can be an alkyl group, optionally substituted with an alkenyl, alkynyl, aryl, aralkyl, cycloalkyl, halogenated alkyl, alkoxy or heterocycloalkyl group. Suitable alkoxy groups include methoxy, ethoxy, n-propoxy, i-propoxy, n-butoxy, i-butoxy, sec-butoxy, tert-butoxy cyclopropoxy, cyclohexyloxy, and the like.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃ -C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁ -C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

The term “co-administration” or “co-administering” refers to administration of a compound disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

“Inhibiting” refers to inhibiting the full development of a disease or condition. “Inhibiting” also refers to any quantitative or qualitative reduction in biological activity or binding, relative to a control.

Small organic molecule: An organic molecule with a molecular weight of about 1000 daltons or less (for example about 900 daltons or less, about 800 daltons or less, about 700 daltons or less, about 600 daltons or less, about 500 daltons or less, about 400 daltons or less, about 300 daltons or less, about 200 daltons or less, or about 100 daltons or less). In some examples, a small organic molecule has a molecular weight of about 100-1000 daltons, about 200-900 daltons, about 300-700 daltons, about 200-500 daltons, or about 400-700 daltons.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. A “prophylactic” treatment is a treatment administered to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters.

In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins. Particular examples of the presently disclosed compounds may include one or more asymmetric centers; thus the compounds described can exist in different stereoisomeric forms. Accordingly, compounds and compositions may be provided as individual pure enantiomers or as stereoisomeric mixtures, including racemic mixtures. In certain embodiments the compounds disclosed herein may be synthesized in or may be purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center(C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁸F, etc

### Overview

Disclosed herein is the druggability of the parathyroid hormone (PTH) type 1 receptor (PTHR), a prototypical class B GPCR and an essential target for the treatment of bone and mineral ion disorders. By employing computational approaches including elastic network model (ENM)-based methods, druggability simulations in the presence of drug-building fragments, pharmacophore modeling and virtual screening with experimental approaches, druggable sites located within the EC vestibule of PTHR are identified. Further comprehensive cell-based signaling analysis of one of these sites uncovered small molecule allosteric modulators of PTHR that reduce and bias subcellular location and duration of cAMP signaling in cultured cells and reduce calciotropic activities in mice in response to PTH, and thus makes these compounds promising pharmacological tools for development of therapeutics against hyperparathyroidism.

PTH is vital for maintaining normal Ca²⁺, phosphate, and vitamin D levels in the body (blood and circulation fluid), and bone turnover. These biological hallmarks are collectively mediated via heterotrimeric Gs and Gq proteins activated in response to PTHR, a class B GPCR mainly expressed in bone and kidney cells. Disturbances in the maintenance of normal mineral-ions due to over-secretion of PTH by the parathyroid glands and PTHR hyperactivity result in hyperparathyroidism, a primary cause of kidney stones and a trigger of osteoporosis (loss of bone mass), two detrimental and costly endocrine diseases affecting the public health. Current pharmaceutical therapies for the treatment of PTH hypersecretion, including calcimimetics (such as cinacalcet, an allosteric activator of the calcium-sensing receptor), calcitriol (active form of Vitamin D) and its analogs to lower serum PTH level, are limited by patients' intolerance, gastrointestinal toxicity, and hypocalcemia also associated with the risk of parathyroidectomy (surgical removals of parathyroids glands). The negative modulation of PTH-induced PTHR signaling is thus a desirable property for drug candidates investigated to treat bone and mineral diseases linked to PTHR hyperactivity, either intrinsic or caused by PTH hypersecretion.

To overcome the limitations of PTH efficacy on skeletal health and address the important gap in the treatment of PTHR hyperactivity, several attempts have been previously made to discover orally available small molecule PTHR ligands leading to identification of three molecules, AH-3960, SW106 and PCO371. The former two molecules were characterized as weak agonist (AH-3960) and weak antagonist (SW106), both acting as orthosteric PTHR ligands. However, their studies were discontinued due to too weak affinities and efficacies. The third molecule (PCO₃₇₁) is currently in phase 1 clinical trial as a potential orally available drug candidate for hypoparathyroidism treatment. It is characterized as an orthosteric agonist of PTHR. The compounds identified herein are unique by their mode of action: they bind to an allosteric site on the extracellular vestibule of the PTHR and negatively modulate receptor signaling induced by PTH in the cytoplasm. As negative modulators, they are candidates to hyperparathyroidism treatment, and as allosteric molecules they have a key advantage over orthosteric ligands in that they modulate endogenous PTH actions and their effects are saturable.

The PTHR is an indispensable class B GPCR regulating blood levels of vitamin D (VitD), calcium and phosphate ions in the body, and bone turnover. Disturbances in the maintenance of normal mineral-ion and VitD balance result in kidney stones and osteoporosis. Furthermore, endocrine diseases of PTHR hyperactivity that include primary or secondary hyperparathyroidism (caused by excess PTH), humoral hypercalcemia of malignancy (caused by excess PTH-related peptide, PTHrP), and Jansen's metaphyseal chondrodysplasia (JMC, caused by constitutive PTHR activity) lead to significant and costly pathologies affecting the public health.

No pharmaceutical therapy is available for the treatment of PTHR hyperactivity, and the only bone-building agents targeting the PTHR to treat osteoporosis are PTH(1-34) (teriparatide) and the modified PTHrP(1-34) (abaloparatide), which have limited efficacy, short plasma lifetimes, and restricted long-term use due to the risk of developing osteosarcoma. In addition, due to their peptidic nature, these existing therapies must be administered via frequent injections often limiting patients' adherence. Therefore, orally available PTHR-targeting small molecule drugs are highly desirable. Furthermore, rather than designing orthosteric drugs that may have promiscuous effects, we focused on designing allosteric drugs that would specifically modulate selected interactions of the PTHR upon altering its structural mechanics. The approach, referred to as ‘allo-targeting’ is becoming increasingly important in drug discovery and development for designing efficacious therapies.

Disclosed herein are compounds that negatively modulate signaling and in vivo function of the PTHR. These compounds act as negative allosteric modulators of PTHR signaling. For example, as described below, when tested in vivo, compound Pitt12 caused significant reduction in the effects of PTH on serum Ca²⁺ and Pi levels in mice, showing that Pitt12 can act as a negative modulator of PTHR function in vivo.

Also disclosed herein is a computational protocol comprised of several methods and tools developed for the identification of allosteric druggable sites in PTHR. The computational protocol involves the integrated use of four modules: (i) essential site scanning analysis (ESSA) to predict the protein sites whose perturbation could elicit cooperative/allosteric responses thus indicating potentially druggable sites; (ii) DruGUI analysis implemented in the ProDy API that is based on the generation and analysis of molecular dynamics (MD) trajectories for the target protein structure in the presence of probe molecules. Probe molecules are small organic molecules (e.g. acetate, imidazole) that are building blocks of most of FDA approved drugs. They help identify protein sites with highest affinity to bind particular types of drug-like fragments; (iii) Pharmmaker suite to build pharmacophore model based on DruGUI analysis; and (iv) virtual screening of compound libraries to identify compounds matching pharmacophore model. By applying this approach to another medically important class B GPCR, the glucagonlike peptide-1 receptor (GLP-1R), and we have discovered the highest affinity druggable site, which overlapped with the small molecule binding site that was experimentally identified and characterized for this receptor (ref: doi10.1038/s41589-020-0589-7). Furthermore, the compounds that were predicted by our protocol to bind this site were highly similar to, and their binding poses overlapped with, the experimentally identified small molecule drug candidate.

### Compounds, Pharmaceutical Compositions and Methods of Treating

Disclosed herein are embodiments of methods utilizing a compound, or a pharmaceutically acceptable salt thereof, of formula I:

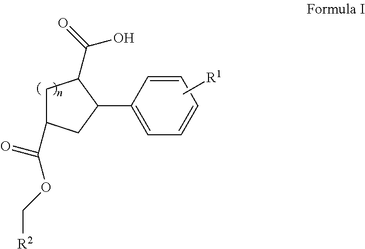

wherein R¹ is hydrogen, alkyl, substituted alkyl, alkoxy, substituted alkoxy, hydroxy, or halogen; R² is phenyl, substituted phenyl,

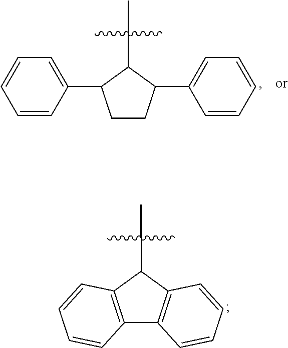 [SMILES: *C1C(c2ccccc2)CCC1c1ccccc1 ; *C1c2ccccc2-c2ccccc21]

and
n is 1 to 3.

In certain embodiments, R¹ is a C₁-C₆ alkyl.

In certain embodiments, R¹ is a C₁-C₈ alkoxy.

In certain embodiments, R¹ is in a para position.

In certain embodiments, R² is

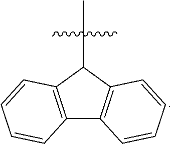 [SMILES: *C1c2ccccc2-c2ccccc21]

In certain embodiments, n is 1.

Also disclosed herein are embodiments of methods utilizing a compound, or a pharmaceutically acceptable salt thereof, of formula II:

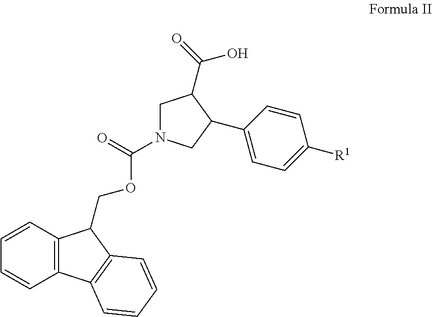

wherein R¹ is hydrogen, alkyl, substituted alkyl, alkoxy or substituted alkoxy.

In certain embodiments, R¹ is a C₁-C₆ alkyl.

In certain embodiments, R¹ is a C₁-C₈ alkoxy.

In certain embodiments, the compound of formula I is

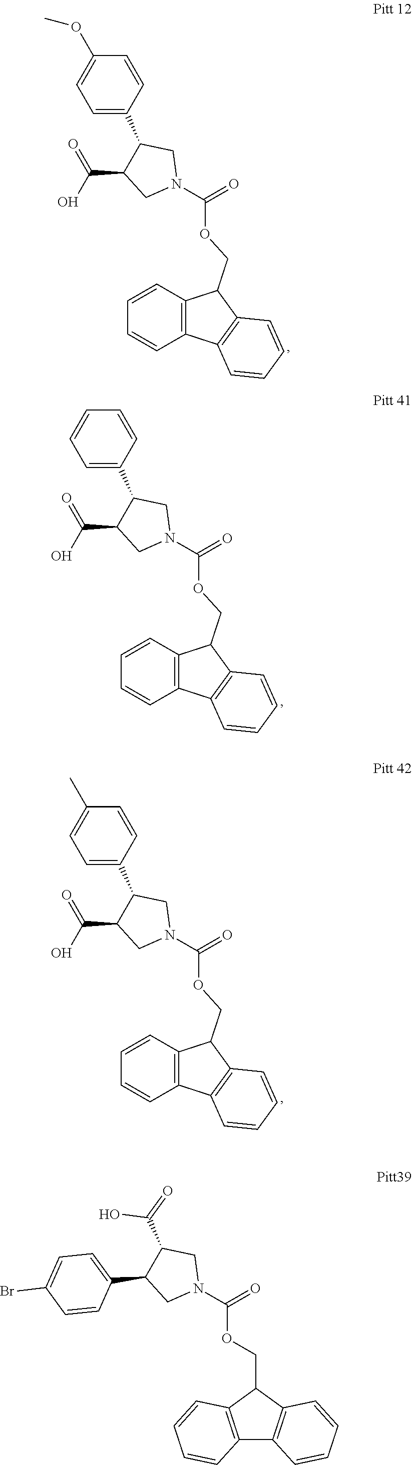 [SMILES: COc1ccc([C@@H]2CN(C(=O)OCC3c4ccccc4-c4ccccc43)C[C@H]2C(=O)O)cc1 ; O=C(O)[C@@H]1CN(C(=O)OCC2c3ccccc3-c3ccccc32)C[C@H]1c1ccccc1 ; Cc1ccc([C@@H]2CN(C(=O)OCC3c4ccccc4-c4ccccc43)C[C@H]2C(=O)O)cc1 ; O=C(O)[C@@H]1CN(C(=O)OCC2c3ccccc3-c3ccccc32)C[C@H]1c1ccc(Br)cc1]

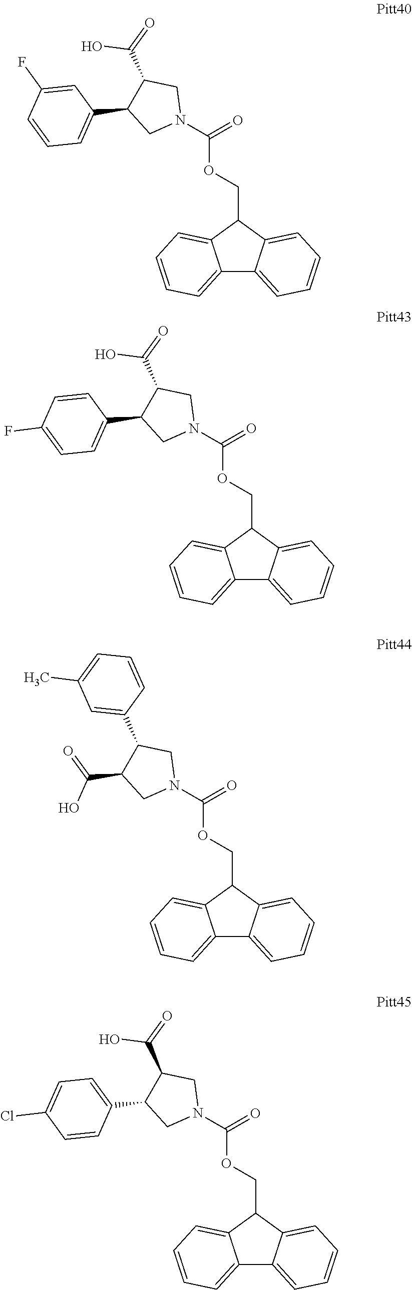

In certain embodiments, the compounds disclosed herein negatively modulate signaling and in vivo function of the PTHR.

In certain embodiments, the compounds disclosed herein act as negative allosteric modulators of PTHR signaling

In certain embodiments, the compounds disclosed herein may be used for treating hyperparathyroidism in a subject. For example, a therapeutically effective amount of the compound may be administered to a subject having, suspected of having, or at risk of developing, hyperparathyroidism.

In certain embodiments, the compounds disclosed herein may be used for treating osteoporosis in a subject. For example, a therapeutically effective amount of the compound may be administered to a subject having, suspected of having, or at risk of developing, osteoporosis.

In certain embodiments, the compounds disclosed herein may be used for treating cancer cachexia in a subject. For example, a therapeutically effective amount of the compound may be administered to a subject having, suspected of having, or at risk of developing, cancer cachexia.

In certain embodiments, the compounds disclosed herein may be used for inhibiting abnormally increased white adipose tissue browning in a subject. For example, a therapeutically effective amount of the compound may be administered to a subject having, suspected of having, or at risk of developing, abnormally increased white adipose tissue browning.

In certain embodiments, the compounds disclosed herein may be used for decreasing the risk of a kidney stone in a subject. For example, a therapeutically effective amount of the compound may be administered to a subject suspected of having, or at risk of developing, a kidney stone.

In certain embodiments, the compounds disclosed herein may be used for reducing the formation of IL-17 secreting Th (T-helper) cells that contribute to the osteocatabolic action (bone destruction) of PTH.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent (such as an anti-retroviral agent) for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### Results

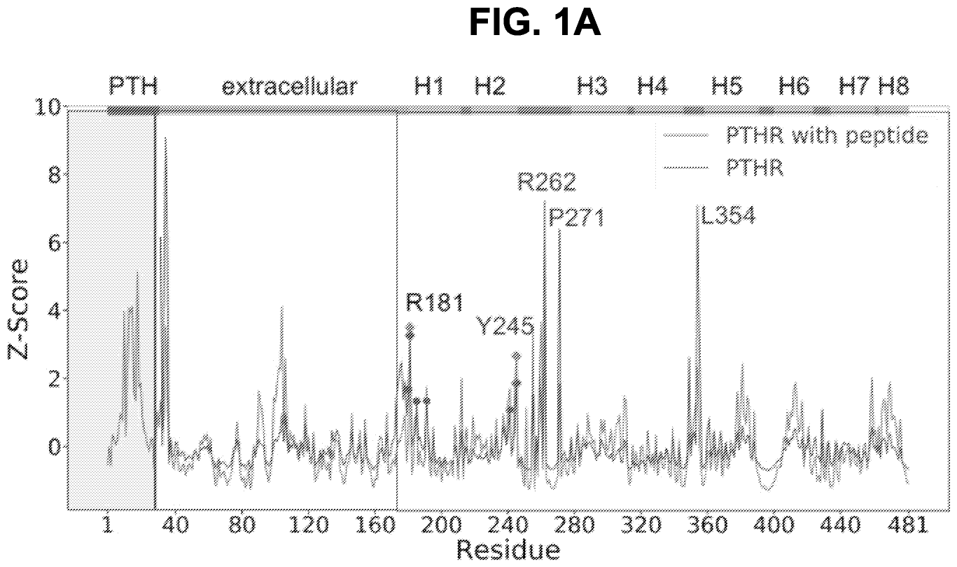

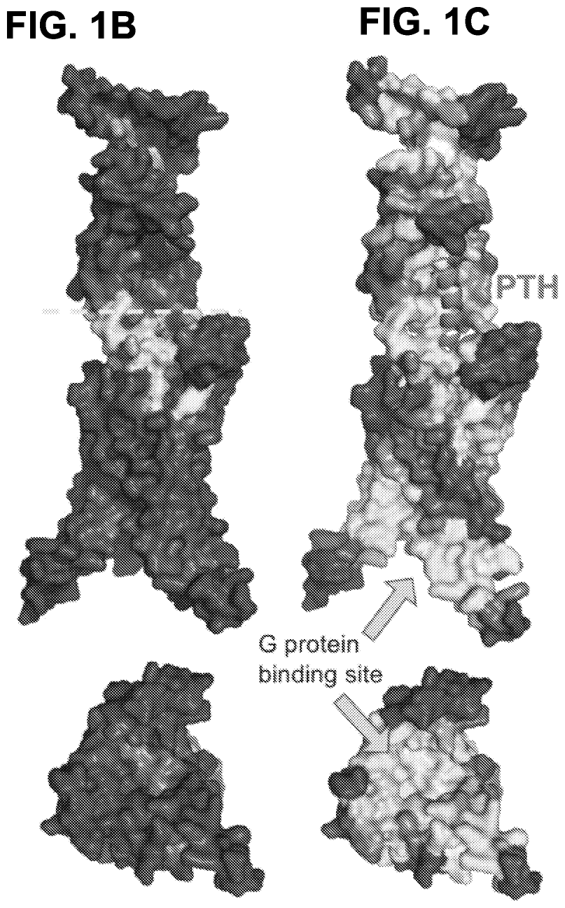

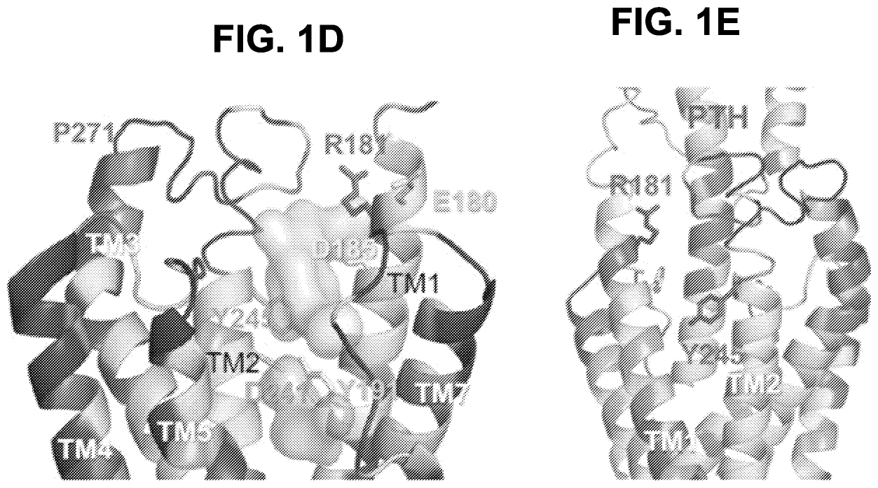

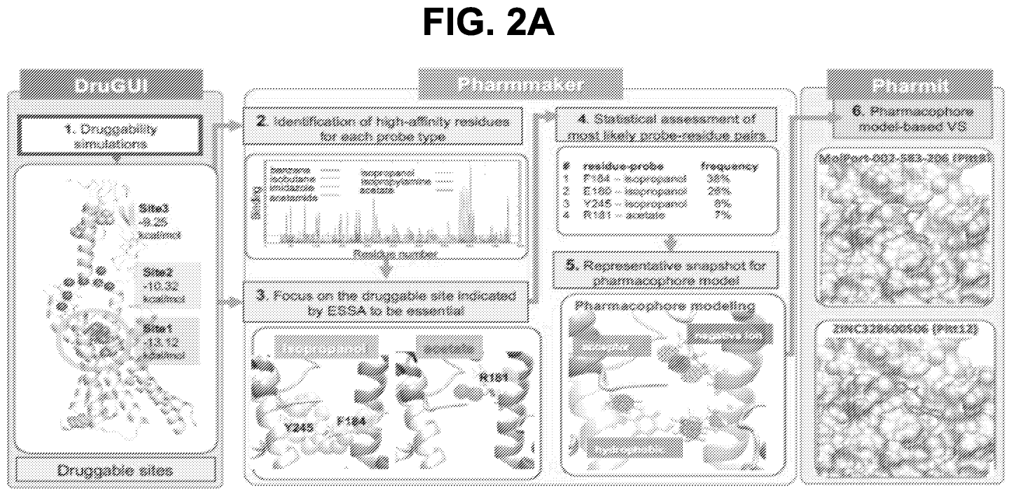

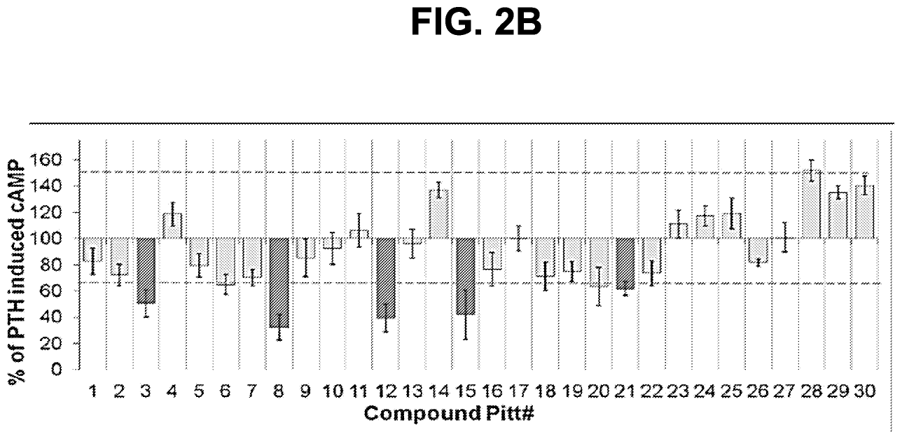

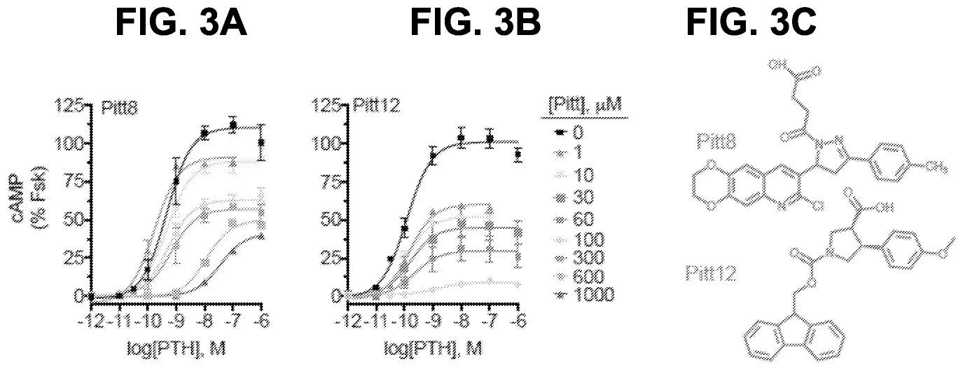

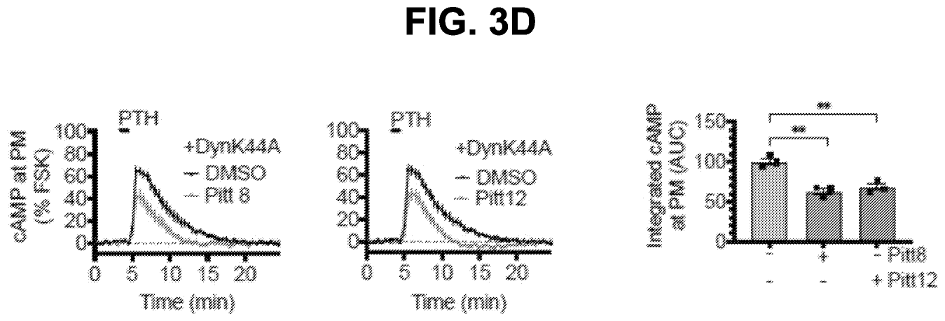

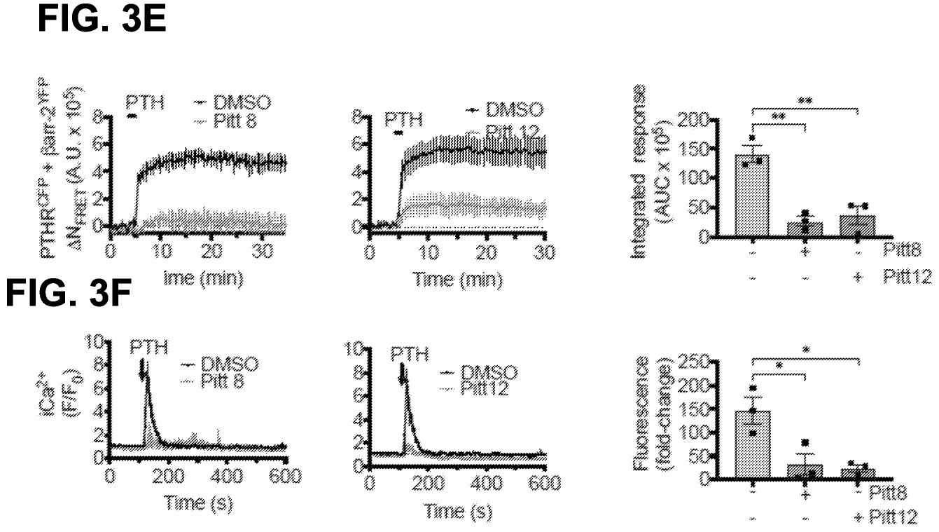

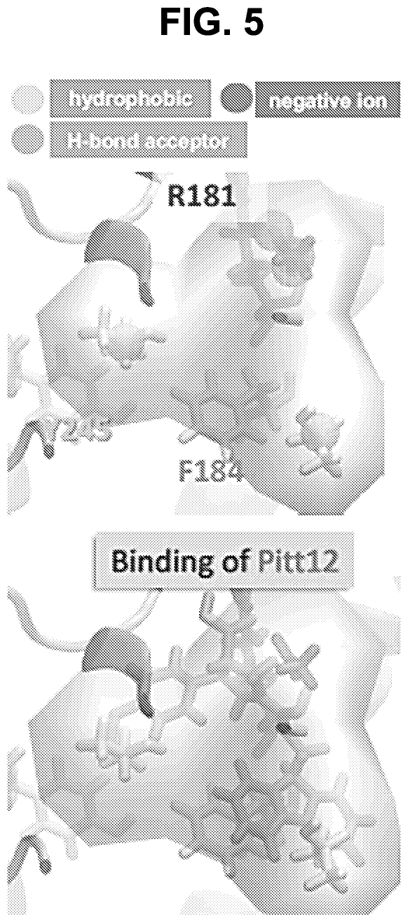

Identification of essential sites with allosteric potential on PTHR. The recently resolved cryo-EM structure of PTHR (PDB id: 6NBF) in its fully activated state bound to a long-acting PTH (LA-PTH) and a dominant-negative Gs heterotrimer, reveal near-atomic details on intra- and intermolecular interaction networks that stabilize the signaling receptor conformation, and provides a structural framework for designing small molecule modulators of PTHR signaling. We used this cryo-EM PTHR structure in its ligand-removed form for modeling and simulations. We initially characterized the PTHR sites whose perturbation could elicit allosteric responses, using a recently developed methodology, Essential Site Scanning Analysis (ESSA). ESSA identifies potential allosteric sites whose change in local density/packing, induced upon small molecule binding, would alter the overall intrinsic dynamics of the protein. Here intrinsic dynamics is defined as the spectrum of modes of motion (i.e., thermal fluctuations) uniquely encoded by the protein fold under physiological conditions, analytically predicted by elastic network models (ENMs). ESSA probes the change in the frequency of the global/essential modes (i.e., the lowest frequency modes that usually underlie cooperative events or allosteric responses upon mimicking small molecule-binding near each residue, scanned over all residues. The peaks in FIG. 1A are such essential sites, color-coded diagrams in FIGS. 1B and 1C. We distinguish residues R181^{1.33b}, Y245^{2.72b} and P271^{ECL1} (superscripts refer to Wootten's class B GPCR nomenclature) in the EC-exposed vestibule lined by the transmembrane (TM) helices TM1 and TM2 (FIG. 1D) (prior to PTH binding). The same site also harbors two pockets detected by FPocket, which further suggests that it might also be druggable (FIG. 1D). Notably, PTH- or LA-PTH-binding to this region (FIG. 1E) and ensuing dramatic change in the dispersion of essential sites (FIG. 1C) underscore the importance of ligand-binding to this site for eliciting an allosteric effect on distal sites, including the G protein binding epitope (see the cytoplasmic face in FIG. 1C bottom).
Druggability simulations and pharmacophore modeling. We next examined the druggability properties of PTHR and studied whether the essential sites identified above (FIG. 1D) were druggable. This was the Step 1 of a 6-step protocol we carried out in silico, schematically described in FIG. 2A. Druggability simulations are molecular dynamics (MD) simulations of the target protein in the presence of explicit water and probe molecules representative of drug-like fragments. We used as probes benzene, isobutane, imidazole, acetamide, isopropanol, isopropylamine, and acetate—derived from statistical evaluation of chemical/functional groups most frequently observed in FDA-approved drugs such as penicillin Simulations revealed three druggable sites, Sites 1-3, distinguished by persistent and/or high concentration of probes (FIG. 2A left diagram). Site 1, composed of E180^{1.32b}, R181^{1.33b}, F184^{1.36b} and Y245^{2.72b}, which coincided with the essential site in FIG. 1D. This site exhibited the highest affinity for the probe molecules and has been selected for pharmacophore modeling. Steps 2 and 3 in FIG. 2A (center diagrams) provide information on the type of probes that bind to Site 1, as well as an upper boundary for ligand-binding affinity at each site using our pharmacophore modeling tool Pharmmaker. Steps 4 and 5 in FIG. 2A describe the construction of a pharmacophore model (PM) accounting for these interactions. To this end, residue-probe interactions are ranked based on their frequency of occurrence within Site 1, and MD snapshots that simultaneously exhibit multiple frequently-observed (entropically favored) interactions are used to construct the PMs.
Virtual screening of library of small molecules and identification of hit compounds. In Step 6 we screened the PM against two libraries of small molecules, ZINC and MolPort, using the Pharmit server. This procedure yielded an ensemble of compounds, 30 of which were selected as top-scoring hits. Two hits, designated as Pitt8 and Pitt12, are shown in FIG. 2A (right diagrams). The computed binding properties of these compounds were further examined by docking simulations and energy minimization. FIG. 5B illustrates the superposition of Pitt12 (ZINC_{328600506/}MolPort 039-313-655) onto the PM. This shows how Pitt12 inserts into the Site 1 identified by both ESSA and druggability simulations, which suggests that it can potentially serve as an allosteric drug, to be investigated next experimentally.
Selection of lead compounds. We performed an initial bioactivity screening of the 30 top-ranking hit compounds identified by the above computational protocol to assess their capacity to modulate the time courses of PTH-induced cAMP production by PTHR stably expressed in HEK-293 cells. Five of these molecules—Pitt3, Pitt8, Pitt12, Pitt15, and Pitt21—displayed a distinguishably high potential to elicit negative effects when co-applied with PTH (FIG. 2B). Notably, none of these molecules exerted any effect on cAMP generation in the absence of PTH, and few of the compounds in this series had moderate positive effects on PTH-induced cAMP generation. Despite their strong inhibitory effects in this screening, compounds Pitt3 and Pitt15 were discarded due to their poor solubility. Thus, we focused on Pitt8, Pitt12 and Pitt 21. We further examined receptor selectivity by testing the effect of Pitt8, Pitt12 and Pitt21 on cAMP production mediated by agonists for the b2-adrenergic receptor (b2AR) and the vasopressin type 2 receptor (V2R). These receptors were chosen for the following reasons: the b2AR expresses in many cell types including those expressing the PTHR, and can increase both PTHR signaling in cells by increasing the duration of cAMP production, and PTHR function in vivo by increasing bone formation in mice in response to intermittent injection of PTH; the V2R shares signaling mechanisms with the PTHR as they are both involved in the regulation of endosomal cAMP production by β-arrestin, G protein subunits G⊖γ, and the retromer complex. Results indicate that only the Pitt21 molecule lacked selectivity by significantly decreasing β₂AR signaling in response to isoproterenol. In contrast, co-application of Pitt8 or Pitt12 had no effect on β₂AR-mediated cAMP production, and the relative selectivity of these compounds was further supported by their lack of effect on vasopressin-mediated cAMP. These results directed us to focus on two candidates for lead compounds, Pitt8 and Pitt12. Next, we found that Pitt8 and Pitt12 compounds had similar negative modulation on cAMP responses mediated by the PTH-related peptide (PTHrP1-36), the N-terminal fragment of other endogenous PTHR agonist. Notably, the modulation of PTH-induced cAMP generation by Pitt8 and Pitt12 was not an artifact of recombinant PTHR overexpression, as similar results were obtained for these compounds in bone and kidney cell lines that endogenously express the receptor. We therefore fully characterized Pitt8 and Pitt12 as lead negative modulators of PTH mediated PTHR signaling.
Pharmacological characterization of selected compounds. The inhibitory binding constant (Kᵢ) of Pitt8 and Pitt12 determined by radioligand binding isotherms is in the μM range for the G-protein uncoupled (R₀) or coupled (R_{G}) states of PTHR. Both Pitt molecules act as negative allosteric modulators of PTHR signaling by markedly decreasing the efficacy (Eₘₐₓ) of PTH to mediate cAMP increase. Pitt8 displayed concentration-dependent effects on both the efficacy (Eₘₐₓ) and potency (EC₅₀) of PTH, whereas Pitt12 induced a marked reduction in efficacy without any significant effects on potency (FIG. 3A, B). While saturable maximal effects observed for Pitt8 reflect expected negative allostery, increasing concentrations of Pitt12 blocked cAMP production by PTH. This pattern is usually associated with non-competitive antagonism (also known as unsurmountable antagonism) that can also result from a strong negative allosteric modulator (NAM) activity of Pitt12 driven by its high degree of negative cooperativity. We next sought to confirm that Pitt8- and Pitt12-mediated alterations in PTHR signaling occur at the level of the ligand-receptor complex by assessing their ability to modulate PTH-induced interaction of G-proteins with the activated receptor. Indeed, FRET-based time courses in cells expressing PTHR C-terminally fused to CFP (PTHR^{CFP}) and mini-GS and -Gq fused to Venus (mGS^{venus} and mGqv^{enus}, respectively) revealed that Pitt8 and Pitt12 cause a decrease in both the kinetics and magnitude of G proteins association with the receptor.

We subsequently determined the actions of small molecules on location bias in PTHR signaling. Biased signaling is a dominant theme in GPCR research, in part, because it has major implications for pharmaceutical development. For the PTHR, the concept is particularly germane, given that the receptor can mediate diverse modes of cAMP signaling, including transient signaling at the plasma membrane or sustained signaling from within endosomes. Such altered modes of signaling likely contribute to the diversity of biological processes regulated by the PTHR, which include the maintenance of calcium homeostasis, bone remodeling, cell differentiation in developing tissues, and the control of renal phosphate excretion. Intriguingly, Pitt8 and Pitt12 exerted selective reduction (≈70%) of endosomal cAMP production with a moderate alteration (≈30%) of plasma membrane cAMP when cells are treated with 1 nM PTH. We found that blockade of receptor internalization by expression of a dominant-negative dynamin mutant (DynK44A) prevented significantly sustained cAMP responses induced by PTH alone (FIGS. 3C, 3D, black traces), while those for PTH when Pitt8 or Pitt12 was coapplied were moderately affected (FIGS. 3C, 3D, pink and green traces). Consistent with the failure of PTH to engage endosomal cAMP in the presence of the selected small molecules, FRET experiments in cells expressing PTHR^{CFP} and β-arrestin-2 fused to YFP (βarr-2^{YFP}) showed that PTHR coupling to βarr2 was markedly impaired (FIG. 3E). In accordance with recent results supporting the dependence of the PTHR-barrestin assembly on the formation of phosphatidylinositol (3,4,5)-triphosphate mediated via Gq activation in response to PTH, Pitt8 and Pitt12 induced noticeable reduction of PTH-mediated release of stored intracellular Ca2+ (FIG. 3F), a result that was corroborated by the reduced Gq coupling to PTHR.

These data suggest that small molecules Pitt8 and Pitt12 can bias the location of cAMP production mediated by PTH, a key feature given that duration and location of PTHR-mediated cAMP responses lead to distinct physiological outcomes, and thus make these compounds promising pharmacological tools for potential therapeutic development.

Refinement and validation of the predicted Pitt12 binding site. Our initial druggability simulations were performed using ligand- and membrane-free PTHR form. To refine the binding pose of Pitt12 in a biologically relevant context and to get insight into mechanism of its action, we performed additional all-atom 200 ns MD simulations using the active state, LA-PTH—bound PTHR structure with receptor embedded in membrane shows predictive interactions and time-evolution of Pitt12 engagement with the peptide ligand and PTHR residues as well as the overall view of the simulation system and bound Pitt12. Pitt12 binds to ligand peptide residues facing TM1 and spanning the peptide's mid-region Q10-R21, overall bridging the LA-PTH and PTHR interaction. Notably, Pitt12 makes persistent contacts with PTHR residues E180^{1.32b} and R181^{1.33b}, identified in the initial druggability simulations. To experimentally validate this prediction, we generated receptor mutants with individual replacement of E180^{1.32b} and R181^{1.33b} to alanines (E180A, R181A), and assessed their sensitivity to Pitt12 modulation of PTH-induced cAMP signaling. While each individual mutation did not alter receptor bioactivity, the mutant receptors lost their ability to respond to negative modulation by Pitt12 suggesting that PTHR residues E180^{1.32b} and R181^{1.33b} are critical for the negative allosteric action of Pitt12.

An intriguing observation predicted by these simulations is that Pitt12 binding alters the mode of LA-PTH interaction with PTHR, in particular shifting the position of peptide's N-terminal tip, which leads to outward displacement of TM5 and TM6 helices. These conformational changes likely disfavor G-protein coupling consistent with negative allosteric action of Pitt12 in time course FRET recodings of PTHR interaction with Gs or Gq.

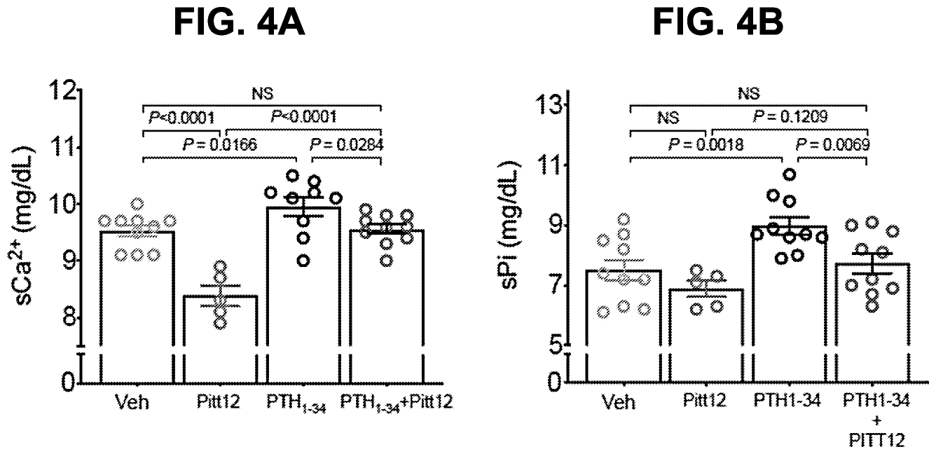

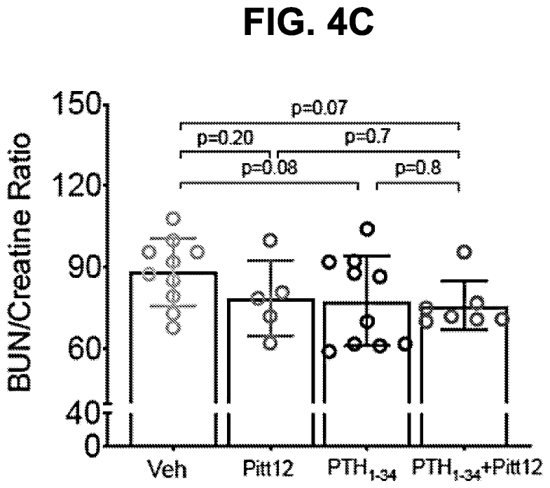

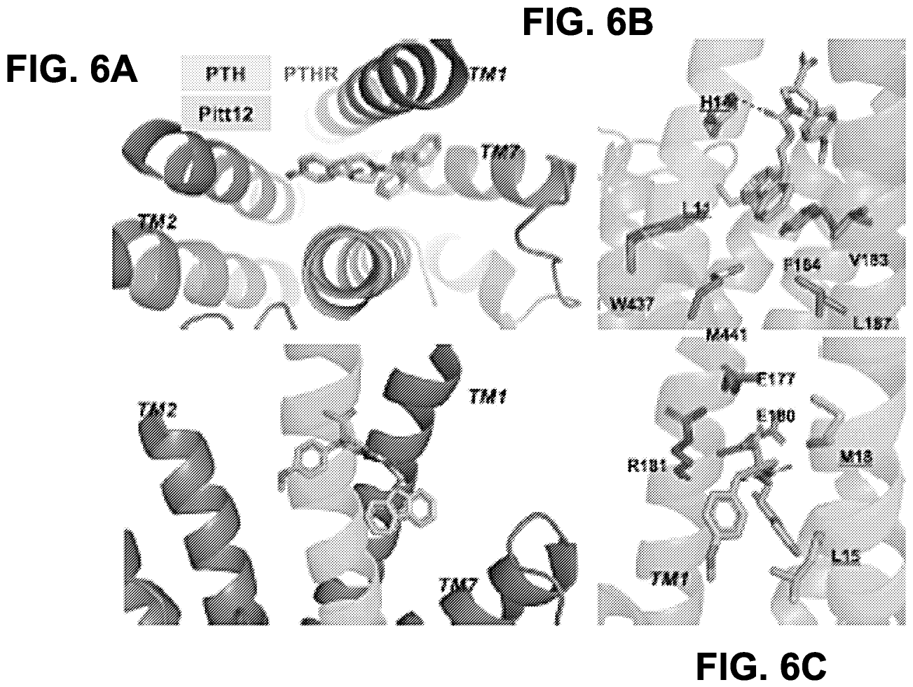

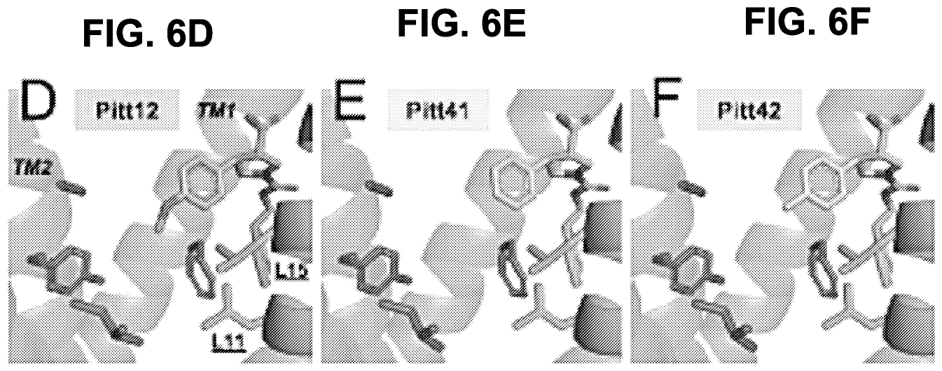

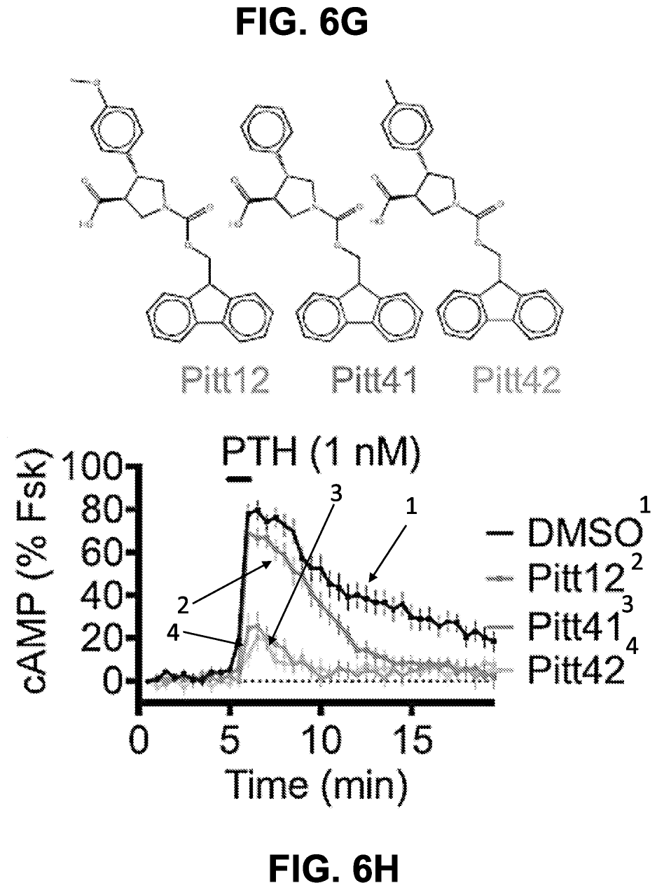

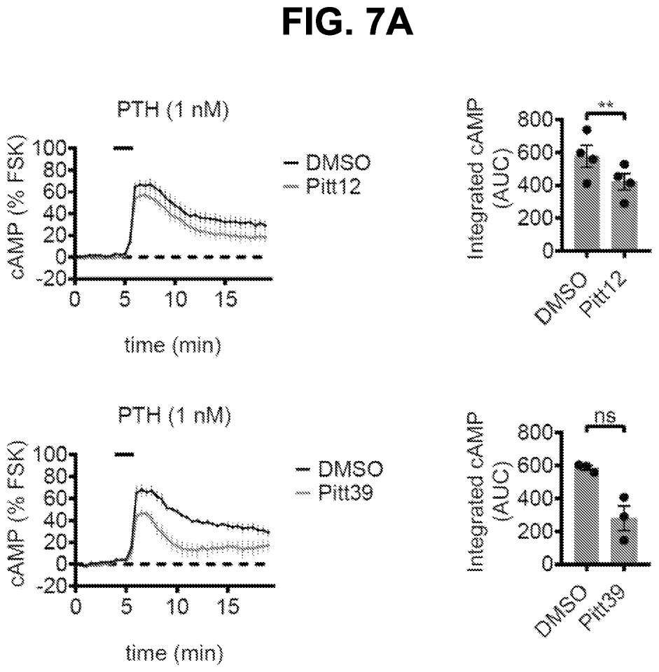

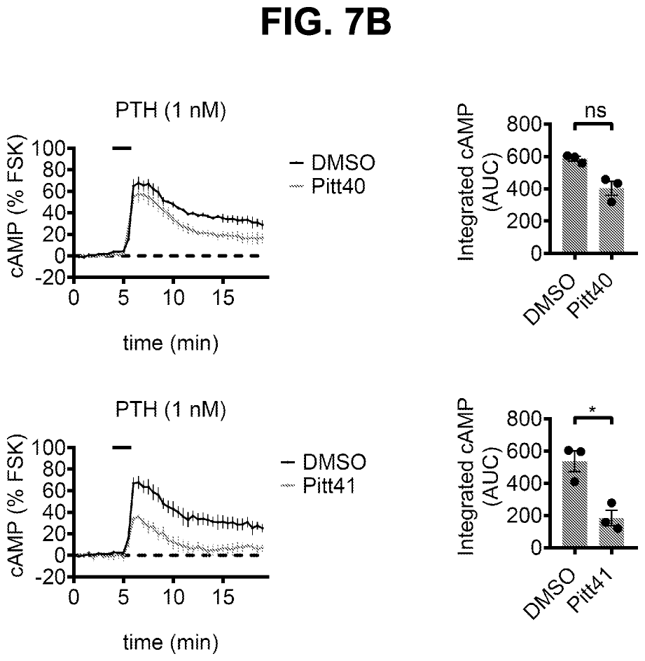

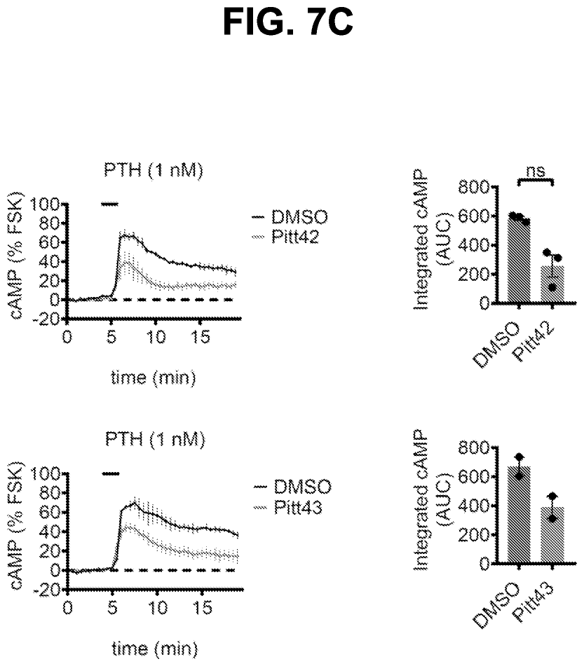

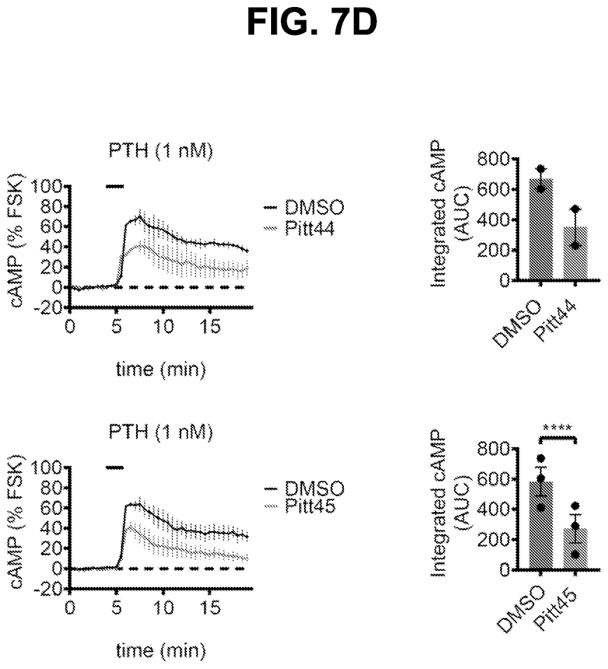

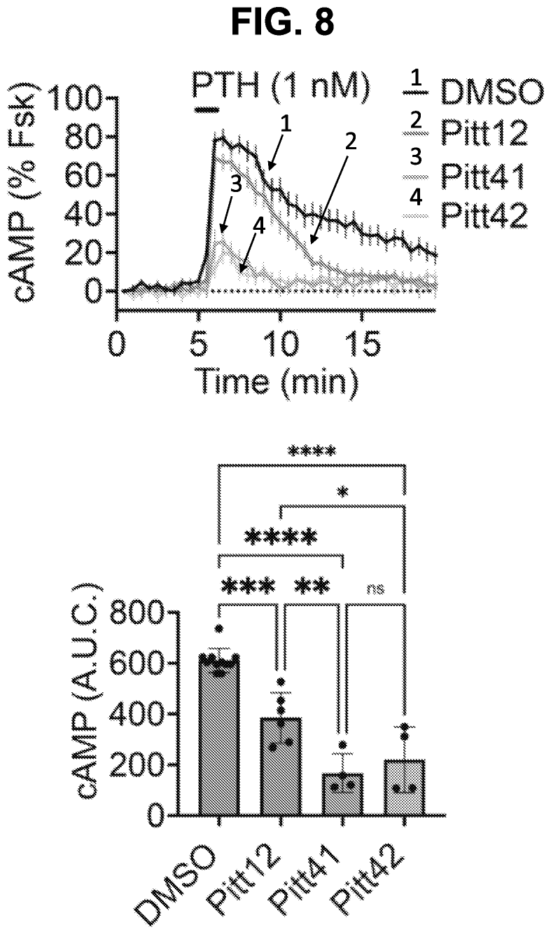

In vivo action of Pitt12. We selected Pitt12 to examine in vivo action of the small molecule. As shown previously, injections of a pharmacological dose of PTH₁₋₃₄ into animals or humans results in acute increase in serum levels Ca²⁺ (sCa²⁺) and phosphate (sPi). We thus examined the ability of Pitt12 to modulate these responses to endogenous and exogenous PTH. Four groups of C57BL/6 mice were randomized and injected with either vehicle, Pitt12 (20 μmol/kg body wt), or PTH1-34 (40 μg/kg body wt) alone or in combination and serum parameters were measured 3 h after injections. Pitt12 alone significantly reduced sCa²⁺ levels and produced a trend of reducing sPi levels, presumably due to its inhibitory action against endogenous PTH (FIG. 4A, B). Consistent with this result, abilities of PTH₁₋₃₄ injection to increase sCa²⁺ and sPi were significantly (P<0.05) reduced by co-injection of Pitt12. The lack of significant changes in BUN (Blood Urine Nitrogen)/creatine ratio indicate normal kidney function and hydration status (FIG. 4C). Overall, our findings support antagonizing actions of Pitt12 against PTHR-mediated effects on mineral homeostasis, potentially by acting as a negative allosteric
regulator for PTHR signaling.
Results with Pitt41 and Pitt42. Analysis of the structural docking of Pitt12 predicted that replacing the methoxybenzene group of Pitt12 by a benzene or methylbenzene moieties could enhance hydrophobic interactions with Leu15 of PTH bond to the PTHR, and thus make a compound with increased binding affinity to the active PTH-bound PTHR structure and improve NAM activity. We tested this theory by comparing the effects of Pitt12 and two compounds, Pitt41 and Pitt42 (FIG. 6G) on PTH-mediated AMP production. Both Pitt41 and Pitt42 were more efficacious than Pitt12 in inhibiting the signaling response mediated by PTH (FIG. 6H), supporting our hypothesis and overall orientation of Pitt12 in the PTH-PTHR structure.

Altogether these findings indicate that the computational pipeline can facilitate the identification of allosteric druggable sites in class B GPCRs and may expedite the discovery of new therapies targeting these medically important receptors.

### EXAMPLES

### Methods

Essential site scanning analysis. ESSA analysis is based on modeling the structure as a Gaussian network model. We used the default cutoff distance of 10 A for defining the inter-residue contact topology using the ProDy application programming interface (API). GNM yields a spectrum of N-1 nonzero modes of motions for a protein of N residues, where each mode is characterized by a frequency and a mode shape described by the respective eigenvalues and eigenvectors of the network connectivity matrix. To mimic the ‘crowding’ induced upon ligand binding, the local density around each residue was increased by adding extra nodes at the positions of heavy atoms other than its a-carbons. The resulting perturbation in the vibrational spectrum of the protein was quantified as a residue-specific z-score (or ESSA score) by evaluating the mean percent shift in the frequency of the ten lowest frequency (also called global) modes for each residue.
Druggability simulations and analyses. Druggability simulations were performed for the resolved PTHR (PDB id: 6NBF) in the presence of probes using the all-atom MD simulation package NAMD with the CHARMM36 force field for proteins, the TIP3P water model, and the CGenFF force field for the probes. The trajectories were analyzed using the DruGUI module of ProDy. The target proteins used for simulations are PTHR (PDB id: 6NBF) and GLP-1R (PDB id: 6VCB). Six independent runs of 40 ns were performed, as recommended. All MD snapshots were superposed onto the reference PDB structure using Cα-atoms and a cubic grid-based representation of the space, with the grid edge size was set to 0.5 A. Probe molecules having non-hydrogen atoms within 4.0 A from protein atoms were considered to interact with the protein. For each probe type, the individual occupancy of grids was calculated using their centroids. We evaluated the occupancy of each probe for a given voxel. High occupancy voxels, called hot spots, within a distance of 5.6 A were merged and druggable sites were defined by clusters of at least six such hot spots. The highest affinity site (Site 1) was further analyzed further to build a PM. The same protocol was adopted for GLP-1R (PDB id: 6VCB.
Pharmacophore modeling. A PM for Site 1 was built using Pharmmaker (see yellow box in FIG. 2A). We identified the residues involved in high affinity interactions with each probe type (Step 2), and also identified highly druggable spots for each probe type (shown in spheres in the figure) within Site 1 (Step3). Residue-probe interactions were rank-ordered based on their frequency of occurrence within Site 1 at the druggable spots in multiple runs (entropic contribution, Step 4). In Steps 3 and 4 in FIG. 2A, we displayed focusing on high affinity residues and probe types we used for PM although we considered all the high affinity residues and probe types at Site 1. Snapshots that exhibited the highest number of the most frequently occurring interaction pairs (e.g., F184^{1.36b}-isopropanol, Y245^{2.72b}-isopropanol, and R181^{1.33b}-acetate) were selected as template to construct PMs (Step 5). The PM was screened against ZINC and MolPort libraries using Pharmit (Step 6). Further refinement of Pitt12 was performed using all-atom MD. We built a system of LA-PTH-bound PTHR (PDB id 6NBF) embedded in POPC lipid membrane and employed simulation steps, using CHARMM-GUI bilayer builder. MD simulation of 200 ns was performed using the package NAMD with the CHARMM36m force field. Data visualization was performed using PyMOL 1.8.6. (Schrodinger L. The PyMOL Molecular Graphics System, Version 1.8.4.2. (2016)).
Cell culture. Cell culture reagents were obtained from Corning (CellGro). Human embryonic kidney (HEK293; ATCC, Georgetown, DC) cells stably expressing the recombinant human PTHR were grown in selection medium (DMEM, 5% FBS, penicillin/streptomycin 5%, 500 μg/ml neomycin) at 37° C. in a humidified atmosphere containing 5% CO₂. Primary calvarial osteoblast (Ob) cells were isolated and cultured as previously described; renal proximal tubule epithelial cells (RPTEC) were grown in DMEM/F12 supplemented with 5 pm triiodo-L-thyronine, 10 ng/ml recombinant human epidermal growth factor, 25 ng/ml prostaglandin E1, 3.5 μg/ml ascorbic acid, 1 mg/ml insulin, 0.55 mg/ml transferrin, 0.5 μg/ml sodium selenite, 25 ng/ml hydrocortisone) plus 1% penicillin and streptomycin. For transient expression, cells were seeded on glass coverslips coated with poly-D-lysine in six-well plates and cultured for 24 hours prior transfection with the appropriate cDNAs using Fugene-6 (Promega) or Lipofectamine 3000 (Life Technologies) for 48-72 h before experiments.
Peptides and small molecules. PTH(1-34) and PTHrP(1-36) were purchased from Bachem. The peptide was resuspended in 10 mM acetic acid to make 1 mM stock solution. Forskolin (#344270) was purchased from EMD-Millipore. Small molecules were purchased from MolPort.
Receptor mutagenesis. We used Genewiz service to generate Human influenza hemagglutinin (HA)-tagged human PTHR mutants carrying E180A (HA-PTHRE180A) and R181A (HA-PTHRR181A), individual mutations.
Time-course measurements of cAMP production, and PTHR recruitment of β-arrestin in single live cells. Cyclic AMP was assessed using FRET-based assays. Cells were transiently transfected with the FRET-based biosensors, Epac1-CFP/YFP for measuring cAMP and PTHR-CFP with βarr2-YFP for measuring arrestin recruitment. For inter-molecular FRET experiments, we optimized expression conditions to ensure the expression of fluorescently labeled proteins was similar in examined cells by performing experiments in cells displaying comparable fluorescence levels. Measurements were performed and analyzed as previously described. In brief, cells plated on poly-D-lysine coated glass coverslips were mounted in Attofluor cell chambers (Life Technologies), maintained in Hepes buffer containing 150 mM NaCl, 20 mM Hepes, 2.5 mM KCl and 0.1-10 mM CaCl2, 0.1% BSA, pH 7.4, and transferred on the Nikon Ti-E equipped with an oil immersion 40× N.A 1.30 Plan Apo objective and a moving stage (Nikon Corporation). CFP and YFP were excited using a mercury lamp. Fluorescence emissions were filtered using a 480±20 nm (CFP) and 535±15 nm (YFP) filter set and collected simultaneously with a LUCAS EMCCD camera (Andor Technology) using a DualView 2 (Photometrics) with a beam splitter dichroic long pass of 505 nm. Fluorescence data were extracted from single cell using Nikon Element Software (Nikon Corporation). The FRET ratio for single cells was calculated and corrected as previously described (42). Individual cells were perfused with buffer or with the ligand for the time indicated by the horizontal bar.
Concentration-response curves for cAMP. HEK293 cells stably expressing human PTH type 1 receptor (hPTHR) and pGloSensor™-22R were seeded in white 96-well plates coated with poly-D lysine at a density of 1.5×104 cells/well. Cells were maintained at 37° C. and 5% CO₂ overnight. For the assay, cell media was replaced by 160 μl Gibco-CO₂ Independent Medium (Fisher Scientific Cat: 1804508) containing 1 mM glucose, 450 μg/ml D-luciferin (Biotium Cat:10101, Lot: 14L0408) and 0.5 mM IBMX (Cayman chemical, item No.13347, CAS No. 28822-58-4), and were incubated 60 mM at room temperature for loading with luciferase. Before drug addition, background time luminescence was recorded at 560 nm in a SpectraMax Paradigm Multiple-Mode microplate reader (Molecular Devices) for 5 mM. The plates were removed from the instrument, a range of PTH1-34 concentrations with or without Pitt molecules (in Gibco-CO2 Independent Medium containing, 1 mM Glucose and 0.5 mM IBMX) were added at different concentrations to a final volume of 200 μl, and luminescence was again recorded in kinetic mode for 45 mM. Peak luminescence or the area-under-curve (auc) of the time course of luminescence response for each PTH1-34 concentration were determined and used to generate concentration-response curves. Data were normalized to the response mediated by of 100 μM forskolin. Curves were fit using GraphPad Prism 8.0 to determine potency (EC₅₀) and maximum efficacy (Emax) of the PTH1-34 response.
Intracellular Ca²⁺ measurements. Changes in intracellular calcium [Ca²⁺]i were measured by loading cells with the live cell calcium sensor Fluo-4 according to manufacturer's directions (Molecular Probes, Carlsbad, CA). Cells were imaged at 2 s intervals using an inverted epifluorescence microscope as previously described
Radioligand receptor binding. Competitive binding to the G protein-dependent and G-protein-independent conformations of PTHR was assessed using membranes prepared from HEK-293-derived GP-2.3 cells (stable expression of hPTHR) and either [¹²⁵I]-M-PTH(1-15) and [¹²⁵I]-LAPTH as tracer radioligands respectively, the latter reaction contained GTPgS (10 μM) as previously described. In brief, reactions (230 μl) were assembled in 96-well vacuum filtration plates (MultiScreen; 0.65 μM, Durapore HV, Millipore Corp., Millford, MA) and contained membrane assay buffer (20 mM HEPES pH 7.4, 0.1 M NaCl, 3 mM MgSO₄, 20% glycerol, 3 mg/ml bovine serum albumin (VWR #97068), 1× protease inhibitor cocktail (SIGMA cat #P8340) radioligand (˜25,000 cpm/well), various concentrations of unlabeled ligand, and membranes (80 μg/ml), added last to start the reactions. Reactions were incubated at room temperature for 90 minutes and terminated by vacuum filtration followed by two rinses of the filters with membrane assay buffer; the filters were then detached, and counted for gamma irradiation. Nonspecific binding was determined using 0.5 μM PTH1-34. Specific bound radioactivity was plotted vs ligand concentration using a four-parameter sigmoidal dose-response equation.
Photometric FRET recordings of G-protein interactions. FRET experiments were performed as previously described. In brief, cells grown on glass coverslips were maintained in buffer A (137 mM NaCl, 5 mM KCl, 1 mM MgCl₂, 20 mM HEPES, pH 7.4) at room temperature and placed on a Zeiss inverted microscope (Axiovert 200) equipped with an oil immersion X100 objective and a dual emission photometric system (Till Photonics, Germany). Cells were excited with light from a polychrome V (Till Photonics). To minimize photobleaching, the illumination time was set to 5-15 ms applied with a frequency between 1 and 20 Hz. Individual cells were perfused with buffer, PTH1-34 alone or in combination with small molecule for the time indicated by the horizontal bar. The emission fluorescence intensities were determined at 535±15 and 480±20 nm (beam splitter dichroic long-pass (DCLP) 505 nm) upon excitation at 436±10 nm (DCLP 460 nm) and were corrected for the spillover of CFP into the 535-nm channel, the spillover of YFP into the 480-nm channel, and the direct YFP excitation to give a corrected FRET emission ratio FCFP/FYFP. Changes in fluorescence emissions due to photobleaching were systematically subtracted.
Mouse Studies. To test the impact of Pitt12 and PTH (PTH1-34) (Bachem, Torrance, CA, Cat #H-5460) on serum Ca²⁺ (sCa) and phosphate (sPi) parameters, 3-month-old male C57BL/6J (C57/B6) mice (Jackson Laboratory, Stock No: 000664) were ear-tagged for identification, randomly assigned to groups, and injected with the drugs (40 μg/kg bogy weight for PTH, 20 μmole/kg for Pitt12). Pitt12 was injected 15 minutes before PTH1-34. In this study, blood was sampled via retroorbital route under isoflurane anesthesia 3 hrs after the drug injection. sCa²⁺and sPi levels were assessed by an ACE Axcel bioanalyzer (Alfa Wassermann, West Coldwell, NJ). All mice were kept in a climate-controlled room (22° C.; 45-54% relative humidity) with a 12-hour light/12-hour dark cycle. Water and standard chow (1.3% calcium and 1.03% phosphate) were given ad libitum. The animal experiments were performed according to procedures approved by the Institutional Animal Care and Use Committee of the San Francisco Department of Veteran Affairs Medical Center (Protocol numbers: 18-017). Mice were acclimated with local environment for 2 weeks before randomization and the experimental procedure. Animal numbers were estimated by a power analysis using the following parameters: standard deviation=15% based our prior studies testing the effects of PTH1-34, two-sided test, p value=0.05, and power of the test=0.8. Additional 20% of mice were included to account for potential fatality due to the drug treatments. The experiments were repeated with 2 different batches of mice that were acquired 2 months apart.
Statistical analysis. Data were processed using Excel 2013 (Microsoft Corp., Redmond, WA) and GraphPad Prism 7.0 or 8.0 (46). Data are expressed as mean±s.e.m. or s.d. Binding or cAMP data from concentration—response assays were analyzed by using a sigmoidal dose-response model with variable slope. Paired data sets were statistically compared by using Student's t test (two-tailed) assuming unequal variances for the two sets. Statistical analyses were performed using two-way ANOVA for in vivo data.

In view of the many possible embodiments to which the principles of the disclosed invention may be applied, it should be recognized that the illustrated embodiments are only preferred examples of the invention and should not be taken as limiting the scope of the invention.

1. A method for treating hyperparathyroidism, osteoporosis or cancer cachexia in a subject, comprising administering to a subject in need thereof a therapeutically effective amount of a compound, or a pharmaceutically acceptable salt thereof, of formula I: 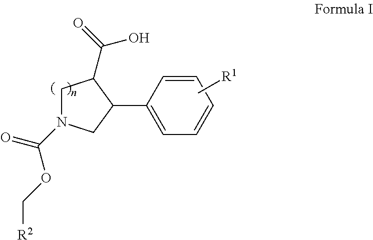
wherein R¹ is hydrogen, alkyl, substituted alkyl, alkoxy, substituted alkoxy, hydroxy, or halogen; R² is phenyl, substituted phenyl, 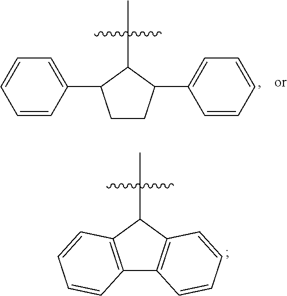 [SMILES: *C1C(c2ccccc2)CCC1c1ccccc1 ; *C1c2ccccc2-c2ccccc21]
and
n is 1 to 3.

2-3. (canceled)

4. A method for inhibiting abnormally increased white adipose tissue browning in a subject, comprising administering to a subject in need thereof a therapeutically effective amount of a compound, or a pharmaceutically acceptable salt thereof, of formula I: 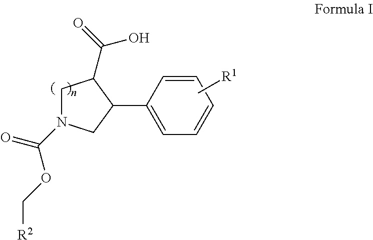
wherein R¹ is hydrogen, alkyl, substituted alkyl, alkoxy, substituted alkoxy, hydroxy, or halogen; R² is phenyl, substituted phenyl, 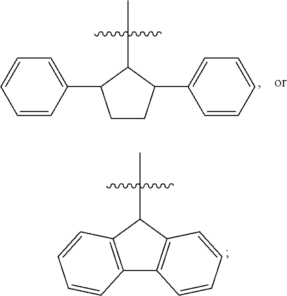 [SMILES: *C1C(c2ccccc2)CCC1c1ccccc1 ; *C1c2ccccc2-c2ccccc21]
and
n is 1 to 3.

5. A method for decreasing the risk of a kidney stone in a subject, comprising administering to a subject in need thereof a therapeutically effective amount of a compound, or a pharmaceutically acceptable salt thereof, of formula I: 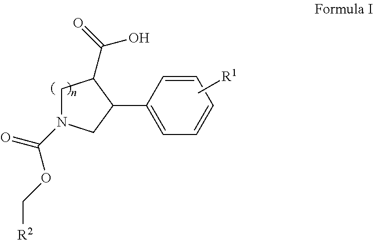
wherein R¹ is hydrogen, alkyl, substituted alkyl, alkoxy, substituted alkoxy, hydroxy, or halogen; R² is phenyl, substituted phenyl, 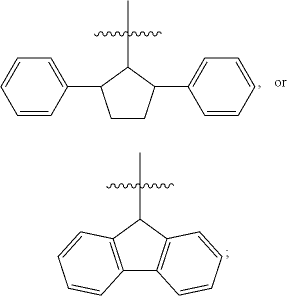 [SMILES: *C1C(c2ccccc2)CCC1c1ccccc1 ; *C1c2ccccc2-c2ccccc21]
and
n is 1 to 3.

6. A method for negatively modulating signaling and in vivo function of a parathyroid hormone (PTH) type 1 receptor (PTHR), comprising contacting the PTHR with a compound, or a pharmaceutically acceptable salt thereof, of formula I: 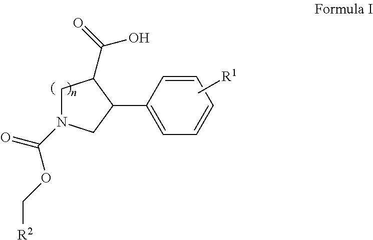
wherein R¹ is hydrogen, alkyl, substituted alkyl, alkoxy, substituted alkoxy, hydroxy, or halogen; R² is phenyl, substituted phenyl, 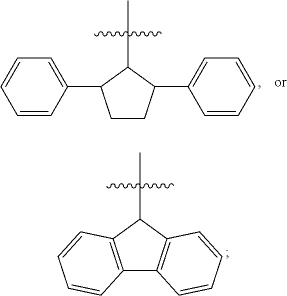 [SMILES: *C1C(c2ccccc2)CCC1c1ccccc1 ; *C1c2ccccc2-c2ccccc21]
and
n is 1 to 3.

7. A method for negatively allosteric modulating signaling of a parathyroid hormone (PTH) type 1 receptor (PTHR), comprising contacting the PTHR with a compound, or a pharmaceutically acceptable salt thereof, of formula I: 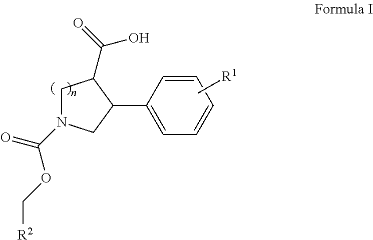
wherein R¹ is hydrogen, alkyl, substituted alkyl, alkoxy, substituted alkoxy, hydroxy, or halogen; R² is phenyl, substituted phenyl, 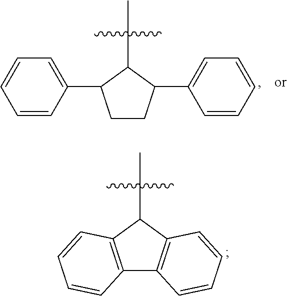 [SMILES: *C1C(c2ccccc2)CCC1c1ccccc1 ; *C1c2ccccc2-c2ccccc21]
and
n is 1 to 3.

8. The method of claim 1, wherein the compound is a compound, or a pharmaceutically acceptable salt thereof, of formula II: 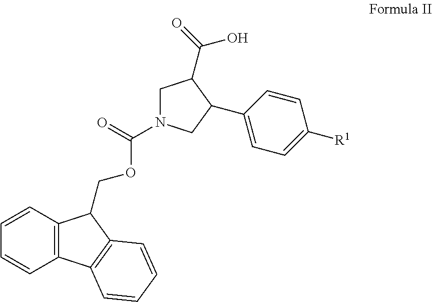
wherein R¹ is hydrogen, alkyl, substituted alkyl, alkoxy or substituted alkoxy.

9. The method of claim 8, wherein R¹ is a C₁-C₆ alkyl.

10. The method of claim 8, wherein R¹ is a C₁-C₈ alkoxy.

11. The method of claim 1, wherein R² is:

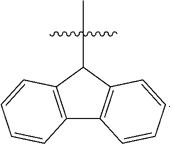 [SMILES: *C1c2ccccc2-c2ccccc21]

12. The method of claim 1, wherein n is 1.

13. The method of claim 1, wherein the compound is

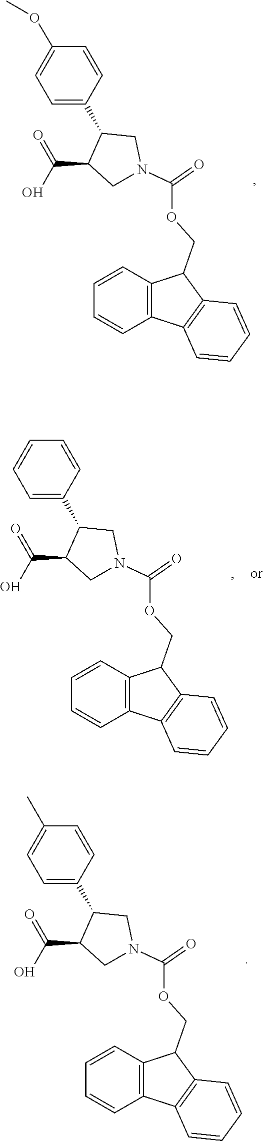 [SMILES: COc1ccc([C@@H]2CN(C(=O)OCC3c4ccccc4-c4ccccc43)C[C@H]2C(=O)O)cc1 ; O=C(O)[C@@H]1CN(C(=O)OCC2c3ccccc3-c3ccccc32)C[C@H]1c1ccccc1 ; Cc1ccc([C@@H]2CN(C(=O)OCC3c4ccccc4-c4ccccc43)C[C@H]2C(=O)O)cc1]

14. The method of claim 1, wherein the compound is

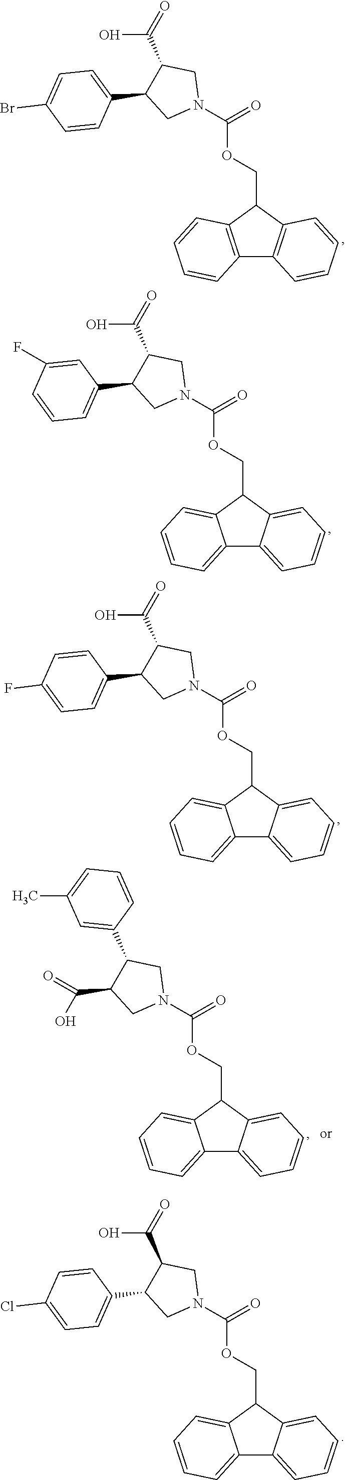

15. A method for treating hyperparathyroidism, osteoporosis, or cancer cachexia, or inhibiting abnormally increased white adipose tissue browning, or decreasing the risk of a kidney stone in a subject, comprising administering to a subject in need thereof a therapeutically effective amount of a negative allosteric modulator of parathyroid hormone (PTH) type 1 receptor (PTHR) signaling.
